(19) United States
(12) Patent Application Publication
(10) Pub. No.: US 2025/0302476 A1
(43) Pub. Date: Oct. 2, 2025

(54) STENT FOR END-TO-END ANASTOMOSIS

(71) Applicant: ConneX Biomedical, Inc., Aurora, CO (US)

(72) Inventors: Jeffrey N. Steinmetz, Avarda, CO (US); Christopher E. Banas, Breckenridge, CO (US)

(73) Assignee: ConneX Biomedical, Inc., Aurora, CO (US)

(21) Appl. No.: 19/232,595

(22) Filed: Jun. 9, 2025

Related U.S. Application Data
(63) Continuation-in-part of application No. 18/458,398, filed on Aug. 30, 2023.
Application No. 18/458,398 is a continuation-in-part of application No. 18/188,393, filed on Mar. 22, 2023.
Application No. 18/188,393 is a continuation-in-part of application No. 18/188,390, filed on Mar. 22, 2023.
Application No. 18/188,390 is a continuation-in-part of application No. 16/739,807, filed on Jan. 10, 2020, now Pat. No. 11,974,748.
(60) Provisional application No. 63/657,528, filed on Jun. 7, 2024.

(51) Int. Cl. A61B 17/11; A61B 17/00; A61F 2/07; A61F 2/915

(52) U.S. Cl. CPC A61B 17/11; A61F 2/07; A61F 2/915; A61B 2017/00862; A61B 2017/00955; A61B 2017/1107; A61B 2017/1132; A61B 2017/1142; A61F 2002/075; A61F 2002/91525; A61F 2002/91575; A61F 2210/0071; A61F 2220/0016; A61F 2220/0025; A61F 2250/001; A61F 2250/0014

(57) ABSTRACT

A device, assembly and method for end-to-side connection to anatomical tissue is disclosed. The device includes a tissue connection stent having a proximal closure to create and maintain a substantial degree of hemostasis. The assembly and method include using a tubular graft member coupled to a plurality of distally projecting tines extending from a distal aspect of the graft that couples the graft to an abluminal wall surface of an anatomic passageway, and the tissue connection stent is then introduced through a central lumen of the graft member, into and through an opening created in the wall of the anatomic passageway, and the tissue connection stent is deployed within the opening and a distal flange of the stent is seated against a luminal wall of the anatomic passageway while the proximal closure is maintained in a closed position until it is deployed to allow fluid flow through the stent.

### CROSS-REFERENCE TO RELATED APPLICATIONS

This application claims priority to U.S. Provisional Patent Application Ser. No. 63/657,528 filed Jun. 7, 2024 and is a continuation-in-part of U.S. patent application Ser. No. 18/458,398, filed Aug. 30, 2023, published Jan. 18, 2024, as U.S. Patent Application Publication No. US 2024/0016495 (the “'495 Publication”), which is a continuation-in-part of U.S. patent application Ser. No. 18/188,393 filed Mar. 22, 2023, published as U.S. Patent Application Publication No. US 2023/0329855, and is a continuation-in-part of U.S. patent application Ser. No. 16/739,807, filed Aug. 12, 2020, now U.S. Pat. No. 11,974,748,issued May 7, 2025. Each of the foregoing is hereby incorporated by reference in their entirety.

### BACKGROUND OF THE INVENTION

The present invention pertains generally to devices and methods for joining an anatomic tissue either to other anatomic tissue or to tubular conduits. Non-limiting examples of uses of the inventive devices and methods include end-to-side anastomosis of a tubular conduit with an anatomic passageway, and/or synthetic or biological graft fixation to soft or hard tissues. Examples of anatomic passageways for which end-to-side anastomosis may be created using the devices and methods of the present disclosure, include, without limitation, blood vessels, lymph ducts, trachea or other airway, esophagus, stomach, small or large intestine, and chambered organs, such as the heart, kidney, or brain.

More particularly, the present invention pertains to an attachment device for coupling an end of the tubular conduit to the side of a portion of the anatomic passageway. Still more particularly, the present invention relates to stent for securing a tubular conduit in an end-to-side anastomosis between the tubular conduit and a portion of the anatomic passageway, such, for example, the aorta or other major blood vessel. Further, the stent of the present disclosure includes a distal flange for anchoring the stent against an inner or luminal wall surface of anatomic tissue and a proximal end configured to allow for delivery in a closed or substantially fluidly or hemostatically sealed state which is then releasable to a diametrically expanded state to allow fluid flow through a central lumen of the stent.

Placement of an end-to-side anastomosis involves penetrating into an anatomic passageway and attaching an external side branching conduit to the anatomic passageway. The side branching conduit is typically a tubular graft that must be affixed in some manner to the anatomic passageway. Access through a wall of the anatomic passageway may be achieved by coring, cutting, or puncturing the wall of the anatomic passageway and may be accompanied by dilation of the opening created in the wall of the anatomic passageway. With blood vessels, in particular, blood flow through the blood vessel needs to be either stopped, such as by clamping or other occlusion, or the access point needs to be sealed in some manner to prevent blood leakage from the blood vessel during and after penetration into the wall of the blood vessel. The stent of the present disclosure is configured to be delivered with its proximal end closed or substantially fluidly sealed, such that the stent may be placed into an unclamped blood vessel or other fluid containing anatomic passageway, without substantial leakage of blood or fluid from the anatomic passageway or blood vessel.

For purposes of clarity and by way of non-limiting example only, reference to a major blood vessel, such as the aorta, may be made herein as an example of an anatomic tissue or passageway onto and into which the assemblies described herein may be deployed. It will be understood, as noted above, that the present invention is useful in forming end-to-side anastomoses with non-vascular anatomic tissues such as tissues of the lymphatic system, gastrointestinal system, renal and urogenital systems, reproductive system, hepatic system, or the like.

Forming an end-to-side anastomosis of a tubular graft to the anastomosis site has been accomplished by a number of different approaches, including suturing a distal end of the tubular graft to an abluminal wall surface of the anatomic passageway (see, e.g., U.S. Pat. Nos. 5,989,287, 6,190,590), stapling the distal end of the tubular graft to either the luminal or abluminal wall surfaces of the anatomic passageway (see, e.g., U.S. Pat. Nos. 5,817,113,6,146,393), securing the distal end of the tubular graft to either the luminal or abluminal wall surface the anatomic passageway by a ring and tines (see, e.g., U.S. Pat. No. 5,951,576, U.S. Patent Application Publication US2003/023251), or by a connecting device such as a connection nipple (see, e.g., U.S. Pat. No. 6,503,258), magnetic rings (see, e.g., U.S. Pat. No. 6,802,847), a stent with an interlocking collar (see, e.g., U.S. Pat. No. 7,063,711), or a stent-like device (see, e.g., U.S. Pat. No. 10,888,412).

None of the heretofore known solutions to affixing a tubular graft to an anatomic passageway in an end-to-side anastomosis, however, have solved or address the problem of limiting fluid flow from the anatomic vessel through the access opening during the anastomosis procedure. The present disclosure addresses this problem by providing a tissue connection stent-like device (hereinafter referred to as a “tissue connection stent”) that is delivered through the tubular graft once the tubular graft is attached to the abluminal surface of the anatomic passageway and before the access opening is created through the wall of the anatomic passageway. This is accomplished by providing a delivery system for the tissue connection stent that serves also as a fluid seal to prevent substantial fluid leakage from the anatomic vessel once the access opening is created and the tissue connection stent delivered and secured within the access opening, then released within the lumen of the tubular graft and secured to the luminal surface of the tubular graft in a substantially fluid tight manner.

The tissue connection stent consists generally of a diametrically expandable lattice structure having a proximal tubular region and a distal flange region. The distal flange region is configured to pass into and through the access opening and seat against the luminal wall surface of the anatomic passageway. The proximal tubular region is configured to project outwardly from the access opening and couple to a distal end of the tubular graft. A covering of a biocompatible material, such as polytetrafluoroethylene (PTFE), thermoplastic polyurethane (TPU), or the like, may be provided on either the entirety or a portion of the tissue connection stent. The proximal tubular region is configured to have a proximal section that is capable of being diametrically constrained so that the proximal end of the proximal tubular region is substantially closed to prevent substantial fluid flow through the tissue connection stent.

The distal flange section of the tissue connection stent also is configured to diametrically expand to provide an entire circumferential distal flange, i.e., subtends an arc of 360 degrees, that projects radially from the distal end of the proximal tubular region. Once the distal flange section is seated in the access opening and against the luminal wall surface of the anatomic passageway, the proximal section is then capable of being released to an open annular state to abut the luminal wall surface of the tubular graft. Release of the proximal section occurs by releasing the diametric constraint to allow fluid flow through the tubular graft and the tissue connection stent at the access site.

The tubular graft is a pliable or semi-pliable tubular graft having a central lumen that opens at proximal and distal ends of the graft. The tubular graft may optionally be configured with a reinforcing distal section that permits angular bending of the graft without the graft kinking to maintain luminal patency of the graft. The reinforced distal section may be achieved by external reinforcing windings around the abluminal surface of the distal section of the tubular graft, by pleats or corrugations at the distal section, varying the wall thickness in circumferential or longitudinal regions of the tubular graft, or by either longitudinal or circumferential regions of different polymer densities in the tubular graft material. Optionally, alignment indicia may be marked on the abluminal wall surface of the tubular graft.

An attachment device consisting generally of an annular ring having a plurality of helically oriented tines projecting axially from a distal end of the annular ring and a plurality of engagement members at a proximal end of the annular ring may be coupled to a distal end of the tubular graft. The helically oriented tines will be delivered and embedded into the wall of the anatomic passageway to secure the tubular graft to the anatomic passageway before the access opening is made. The annular ring may be a unitary ring, or a segmented ring. Where a segmented annular ring is employed, it may be comprised of plural arcuate sections arranged to form a substantially annular ring structure. The plural arcuate sections may be configured to interface with one another in a reciprocally slidable arrangement, an articulating arrangement, or other arrangement where individual or groups of arcuate sections are movable relative to other arcuate sections of the annular ring. The annular ring may further include more than one annular ring structures with each annular ring having helically oriented tines that are oriented in counter-rotating fashion relative to the other annular ring structure. Alternatively, where more than one annular ring is employed, the lengths, configuration, shape, rotational orientation, angles, or spacing of the helically oriented tines may be different in a first annular ring than in a second annular ring.

A graft delivery device may be provided that includes an axial compression sleeve and a driver for rotating the attachment device and the attached tubular graft to embed the helically oriented tines of the graft component into the anatomic tissue. The axial compression sleeve is longitudinally translatable relative to the driver, the graft component, and the attachment device, and is extensible beyond the distal end of the attachment device to axially compress the anatomic tissue and create a substantially planar surface on the anatomic tissue to engage the helically oriented tines into.

A coring tool may also be provided either independently or as a component of the graft delivery device. The coring tool will include a core cutting knife and, optionally, a stabilization device. When a component of the graft delivery device, the coring tool will pass concentrically within the lumen of the tubular graft to access the desired coring site. The optional stabilization device is configured to pass concentrically through the coring tool and attach, at its distal end, to the abluminal wall tissue of the anatomic passageway, with the coring knife concentrically positioned about the distal end of the stabilization device. Once attached to the abluminal wall surface, the coring knife is used to cut the access opening and create a cored tissue plug which is retained on and withdrawn with the stabilization device and the coring tool through the lumen of the graft. An example of the coring tool and stabilization device configurations are described in greater detail in the related '495 Publication which is incorporated by reference.

As described in the '495 Publication, the stabilization device includes a stabilization tubular member having a handle at a proximal end and a coring retention member at a distal end of the stabilization tubular member. The core cutting device includes a coring tubular member having a handle at a proximal end thereof and a core cutting member at a distal end of the coring tubular member. Optionally a flushing port may be provided in either the coring tubular member or the coring tubular member handle. Further, a set screw and set screw receiver opening may be provided in the coring tubular member or the coring tubular member handle to prevent undesired rotation of the core cutting device. The coring retention member penetrates into the anatomical tissue and may be rotated to cut a tissue plug from the anatomic tissue, with the tissue plug being retained on the coring retention member at the distal end of the stabilization tubular member.

˜The stent component includes a stent having a collapsed state and an expanded state,

and having a distal circumferential flange that extends radially outward away from a central longitudinal axis of the stent when the distal circumferential flange is in the expanded state. The stent consists generally of a tubular proximal section and the distal circumferential flange at a distal portion of the stent. As is known in the stent arts, the stent of the stent component may have many different structural geometries. That is that the stent may be made of a single or plural materials, may be made of a single hypotube that is machined into structural members that form the wall surfaces of the stent, may be made balloon-expandable or self-expanding, in whole or in part, may optionally include radiopaque markers, may optionally include drug-eluting capabilities, or such other structural and/or functional features as are known in the stent arts.

The stent may, optionally, be further covered by or encapsulated with, in whole or in part, a graft covering that covers the luminal wall surface and/or abluminal wall surface of the stent. Where provided, the graft covering may cover only the proximal tubular section, only the distal circumferential flange, or both the proximal tubular section and the distal circumferential flange. Where provided, the graft covering preferably extends about an entire circumferential aspect of the portion or portions of the stent which it covers or encapsulates.

The stent component further includes a delivery system for the stent. The stent delivery system may have a stent retaining sleeve from which the stent is deployed by translating the stent relative to the retaining sleeve to impart a controlled release of the distal circumferential flange section separately from the proximal tubular section of the stent. Optionally, the stent delivery system further includes a proximal retention member that is removably coupled with and releasable from a proximal end of the proximal tubular section; the proximal retention member may, optionally, be configured to seal a central lumen of the proximal tubular section in order to prevent fluid flow through the central lumen during the delivery process and maintain a degree of hemostasis. The proximal retention member may be configured in a wide number of ways to both removably couple to the proximal tubular section of the stent, release the stent, and maintain a degree of hemostasis by sealing the proximal tubular section of the stent until the stent is released from the proximal retention member. An example of one configuration of the proximal retention member is to provide a sealing device such as that described in U.S. Pat. No. 9,345,461 in which a coiled sealing element is made of a coiled string element that is engaged with the proximal tubular section of the stent to seal the stent lumen. Once the proximal tubular section of the stent is positioned, the coiled string element is withdrawn, unwinding the coiled string element and deploying the tubular portion of the stent, releasing the seal provided by the sealing device and opening the stent lumen. It will be appreciated by those in the art that various other methods of providing a releasable sealing element in conjunction with the proximal tubular section of the stent are intended and contemplated by the present disclosure.

In one example of an application of the end-to-side anastomosis assembly, the assembly has particular application in coupling an outflow of a circulatory assist device. Examples of circulatory assist devices are ventricular assist devices (“VAD”) and left ventricular assist devices (“LVAD”) that pump blood from the heart and create a blood flow path from the pump to the aorta. Such VAD devices may be centrifugal or axial flow pumps. End-to-side anastomosis assemblies of the present disclosure afford either access to introduce a VAD to the heart and/or allow for fluid flow from a VAD pump output to the aorta.

It will be understood that the end-to-side anastomosis assembly has other non-vascular medical applications to anatomic passageways. Additionally, non-medical applications of the devices and methods are also intended and contemplated by the present disclosure. Further, reference to the medical application of the devices and methods of the present disclosure to circulatory assist devices, such as VADs or LVADs devices, is intended to be a non-limiting example of an application of the devices and methods described herein.

Heart failure is a leading cause of death in developed countries. An estimated 100,000 Americans develop end-stage congestive heart failure each year with a one-year mortality of approximately 50%. There are many etiologies of heart failure. Treatment options depend on the underlying cause and consist of drug therapy, catheter based or surgical interventions for coronary artery disease, and catheter or surgical procedures for valve disease and other lesions. In the past, the only treatment for end-stage non-correctable heart failure was heart transplantation. Approximately 4,200 heart transplant procedures are performed annually in the United States and approximately 7,500 are performed annually world-wide. At any given time, there are approximately 3,000 patients on the heart transplant waiting list in the United States. Consequently, demand for transplantation far outstrips the supply of donor hearts, and it is unlikely that this supply imbalance will improve. Because of the donor supply imbalance, practitioners have developed mechanical VAD systems to support the circulation in patients with heart failure.

Initially, VAD therapy was limited to heart transplant candidates and was intended to bridge patients to heart transplant and improve their baseline health status going into transplant. This strategy is commonly referred to as Bridge to Transplant (“BTT”). As technology improved, VAD outcomes improved, and VAD therapy was extended to the larger population of heart failure patients who are not candidates for transplant. VAD treatment in the latter pool of patients is referred to as Destination Treatment (“DT”). Most patients, regardless of treatment intent, can be supported with a left sided device alone.

The current generation of LVADs in common use are continuous flow devices. The newer continuous flow LVADs are small enough to be implanted entirely within the pericardial space and do not require an intra-abdominal pocket. In general, the pumping inlet mechanism of current LVADs is surgically attached directly to a heart chamber. The outflow end of the pumping mechanism consists of a prosthetic vascular tube graft that is sewn end to side to a major artery-usually the aorta. There are other surgical indications for the attachment of the end of a large prosthetic vascular graft to the side of a major artery. For example, aorta to aorta bypass procedures require end-to-side attachment of a prosthetic graft to the aorta at one end and the aorta at the other end. An alternative example of an application for the end-to-side anastomosis system is in implanting an apical-aortic valve conduit in which a valved conduit is implanted into the left ventricular apex and then a distal end of the valved conduit is joined by an end-to-side anastomosis to the aorta bypassing the aortic valve.

Conventional methods for the surgical attachment, e.g., anastomosis, of the end of a large prosthetic tube graft to the side of a major artery typically involve isolating a segment of the target artery with a side-biting clamp or between two completely occlusive clamps. An opening is created in the target artery, known as an arteriotomy, and the prosthetic tube graft is manually sutured to the arteriotomy in an end to side manner with the arteriotomy opening and a central lumen of the prosthetic tube graft being in fluid flow communication with each other. Suturing methods vary and include running suture techniques, interrupted suture techniques, i.e., using a plurality of individually placed and tied sutures, or a combination of these methods. Conventional suturing methods are time consuming, require clamping of the target vessel which in some cases may be diseased, and can be associated with suture hole bleeding due to suture hole elongation. In contrast, by eliminating the suturing, the assembly of the present disclosure improves hemostasis decreases procedure time, standardizes the procedure so that it is not dependent upon a surgeon's technical abilities, and will facilitate the use of minimally invasive incisions for surgical access to the site for the end-to-side anastomosis.

While the present invention will be described with respect to its use with a VAD procedure and system, those skilled in the art will understand and appreciate that the scope of the present invention is intended not to be limited to VAD procedures and systems but to end-to-side connections between tubular medical grafts, autologous anatomical tubular grafts, heterologous or other biological tubular grafts, and other anatomical structures, such as the gastrointestinal system, biliary system, lymphatic system, urinary system or the like.

### SUMMARY OF THE INVENTION

The currently disclosed devices, assemblies, and method for making an end-to-side connection are well suited to forming attachments between a tubular conduit and an anatomic passageway, such as a major blood vessel.

As a non-limiting example of a use of the disclosed assemblies, there is provided an end-to-side connection assembly configured to be coupled to a wall of a large blood vessel, such as the aorta, to direct blood flow into the vessel or provide access to the lumen of the blood vessel.

Other non-limiting examples may include use of the end-to-side connection assembly to couple other anatomic passageways, such as the small intestine, large intestine, stomach, or rectum, bile ducts, or the like.

It is, therefore, an objective of the present disclosure to provide a stent configured to pass through an access opening in an anatomic passageway, abut against a luminal wall surface of the anatomic passageway, project proximally through the access opening, and have a proximal section that is configured to be optionally diametrically expandable and contractable to open and close the central lumen of the stent after the stent is deployed through the access opening and couple to or abut a luminal wall surface of a tubular graft through which the stent is deployed.

It is a further objective of the present disclosure to provide an end-to-side connection assembly consisting generally of the stent and a tubular graft component. Optionally, the coring and/or stabilization component may also be provided as part of connection assembly.

It is yet a further objective of the present disclosure to provide an end-to-side connection assembly that is particularly well suited for connecting an end of a tubular conduit to a side wall of another tubular conduit to establish a fluid flow connection therebetween.

### BRIEF DESCRIPTION OF THE DRAWINGS

FIG. 1 is a plan view of a tissue connection stent in accordance with the present disclosure.

FIG. 2 is a perspective view of the tissue connection stent in a fully diametrically expanded state and having a partial polymer covering.

FIG. 3A is a perspective view of the tissue connection stent illustrating a proximal end section in a diametrically constrained state and the remainder of the tissue connection stent in a fully diametrically expanded state.

FIG. 3B is a perspective view of the tissue connection stent of FIG. 3A in a fully diametrically expanded state.

FIG. 4A is a perspective view of the tissue connection stent illustrating an uncovered proximal end section in a diametrically constrained state and the remainder of the tissue connection stent being covered in polymer and in a fully diametrically expanded state.

FIG. 4B is a perspective view of the tissue connection stent of FIG. 4A in a fully diametrically expanded state.

FIG. 5A is a perspective view of the tissue connection stent having a full polymer covering and with the proximal end section in a diametrically constrained state using a purse-string tie, with the remainder of the tissue connection stent in a fully diametrically expanded state.

FIG. 5B is a perspective view of the tissue connection stent of FIG. 5A in a fully diametrically expanded state.

FIG. 6 is a perspective view of a tubular graft having a distal reinforced section.

### DETAILED DESCRIPTION OF THE PREFERRED EMBODIMENTS

For purposes of clarity, the following terms used in this patent application will have the following meanings:

The terminology used herein is for the purpose of describing example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the order discussed or illustrated, unless specifically identified as an order of performance. It is also to be understood that additional or alternative steps may be employed.

When an element or layer is referred to as being “on,” “engaged,” “connected,” or “coupled” to or with another element, it may be directly on, engaged, connected or coupled to the other element or layer, or intervening elements or layers may be present. In contrast, when an element is referred to as being “directly on,” “directly engaged to,” “directly connected to,” or “directly coupled to” or with another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below”, or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

“Substantially” is intended to mean a quantity, property, or value that is present to a great or significant extent and less than, more than or equal to total. For example, “substantially vertical” may be less than, greater than, or equal to completely vertical.

“About” is intended to mean a quantity, property, or value that is present at ±10%. Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. In addition, disclosure of ranges includes disclosure of all values and further divided ranges within the entire range, including endpoints given for the ranges.

The use of the terms “a” and “an” and “the” and similar referents in the context of describing the invention are to be construed to cover both the singular and the plural, unless otherwise indicated herein or clearly contradicted by context. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Recitation of ranges of values herein are merely intended to serve as a shorthand method of referring individually to each separate value falling within the recited range, unless otherwise indicated herein, and each separate value is incorporated into the specification as if it were individually recited herein.

References to “embodiment” or “variant”, e.g., “one embodiment,” “an embodiment,” “example embodiment,” “various embodiments,” etc., may indicate that the embodiment(s) or variant(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment or variant, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

The terms “proximal” or “distal” are intended to be relative positional references and are used with reference either to a direction of blood flow relative to a device or device component or with reference to a longitudinal axis of a device or device component. For example, with reference to the graft component, the proximal end of the graft component furthest away from the major vessel or anatomic passageway, whereas the distal end of the graft is the end closest to the major vessel or anatomic passageway.

The term “annular” when used in connection with an element is intended to mean a ring-like structure comprised of either a unitary structure or a discontinuous structure having plural arcuate sections arranged into a ring-like structure.

The term “saddle-shape” when used in connection with an element is intended to mean a generally hyperbolic paraboloid structure.

The term “graft” is intended to refer to any type of polymeric, biological, composite or metal structure.

The term “anatomic passageway” is intended to refer to any anatomical structure having a lumen. Examples of anatomic passageways are blood vessels, the gastrointestinal track, including the esophagus, stomach, small intestine, large intestine, and rectum, or airway passages, such as the trachea and bronchi.

The terms “major vessel” and/or “aorta” as used herein reference specific and non-limiting examples of anatomic passageways. It is intended that the terms “anatomic passageway,” “major vessel,” and/or “aorta” are used interchangeably and synonymously.

The term “flange” is intended to refer to any type of radially extending projection, including, without limitation, a projection that extends less than or equal to 360 degrees relative to the element that the projection extends from. Further, a flange may have a longitudinal component to its projection orientation relative to the element that the projection extends from.

This detailed description of exemplary embodiments references the accompanying drawings, which show exemplary embodiments by way of illustration. While these exemplary embodiments are described in sufficient detail to enable those skilled in the art to practice the disclosure, it should be understood that other embodiments may be realized and that logical changes and adaptations in design and construction may be made in accordance with this disclosure and the teachings herein without departing from the spirit and scope of the disclosure. Thus, the detailed description herein is presented for purposes of illustration only and not for purposes of limitation.

The accompanying Figures illustrate various embodiments of the tissue connection stent 10 and the graft 100 are useful in forming an end-to-side anastomosis. As will be described further hereinafter, the graft 100 has a central lumen 110 and an axial compression component 120 at a distal end of the graft 100. The axial compression component 120 engages and embeds into anatomic tissue to create a putative fluid flow conduit between an anatomic passageway and the central lumen 110 of the graft 100 once an access opening (not shown) is formed in the anatomic tissue. The tissue connection stent 10 is configured to pass through the central lumen 110 of the graft 110, and into the access opening in the anatomic tissue. The tissue connection stent 10 has a first state in which the tissue connection stent 10 is in a diametrically reduced state for delivery into and through the graft 100 and a second state in which the tissue connection stent 10, or sections thereof, are in a diametrically expanded state.

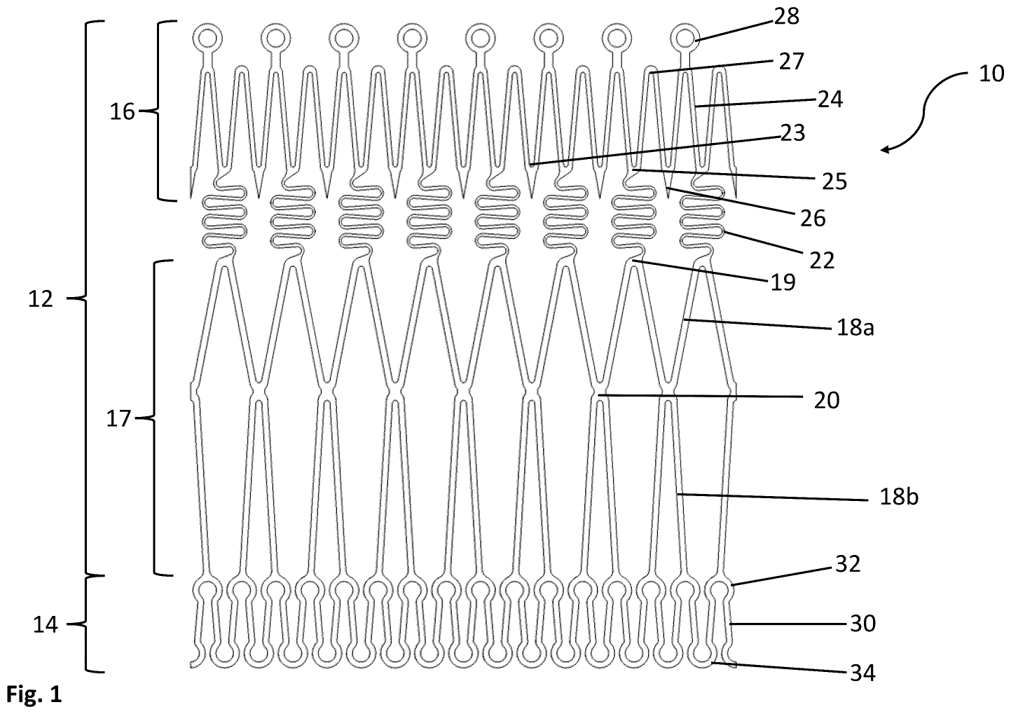

The tissue connection stent 10 is characterized by having a lattice framework a lattice framework defining a proximal tubular section 12, a distal flange section 14 and a proximal end section 16. The proximal tubular section 12 is comprised generally of a proximal end section 16, a distal end section 17, and a plurality of flexion members 22 joining the proximal end section 16 and the distal end section 17 of the proximal tubular section 12, as illustrated in FIG. 1. The distal end section 17 is comprised of a plurality of first strut members 18a forming a first repeating pattern of first strut members 18a, a plurality of second strut members 18b forming a second repeating pattern of second strut members 18b, and a plurality of connection points 20 connecting apices of the first strut member 18a and the apices of the second strut members 18b. The proximal end section 16 is comprised of an undulating pattern of third strut members 24 forming proximal apices 27 and distal apices 25 at juncture points between the plurality of third strut members 24. At least one eyelet member 28 is provided that projects from at least some of the proximal apices 27 of the plurality of third strut members 24, while some of the distal apices 25 of the third strut members 24 are joined to the flexion members 22 and some of the distal apices 25 of the third strut members 24 form barb projections extending distally from the distal apices 25.

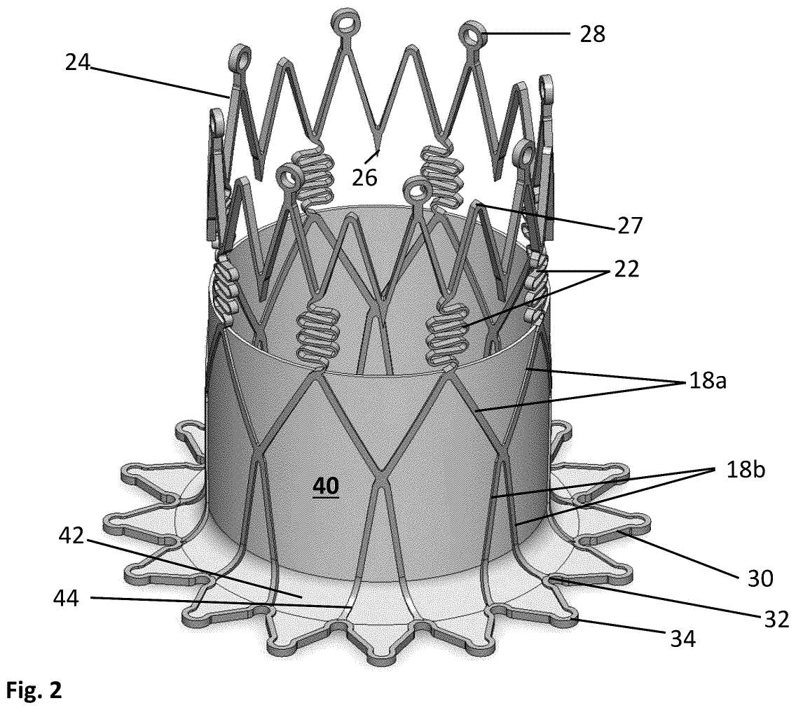

The distal flange section 14 comprises a plurality of undulating fourth strut members 30 having first strain relief members 32 at a proximal end of the fourth strut members 30 and second strain relief members 34 at a distal end of the fourth strut members 30. The first strain relief members 32 and the second strain relief members 34 interconnect adjacent fourth strut members 30 and allow the entire distal flange section 14 to expand diametrically and radially to form the expanded distal flange section 14 when the distal flange section is in the second state of being diametrically expanded. The expanded distal flange section 14, as shown in FIG. 2, circumscribes an entire circumferential axis, i.e., 360 degrees, around a distal end section 17 of the proximal tubular section 12 of the tissue connection stent 10. Distal aspects of the second strut members 18b that join to the fourth strut members 30 are configured to have a section 44 that deforms radially outward to accommodate transition between the distal end section 17 of the proximal tubular section 12 and the distal flange section 14 when they are in their second state of being diametrically expanded.

The fourth strut members 30, the first strain relief members 32 and the second strain relief members 34 form an undulating ring structure that forms the distal flange section 14. The first strain relief members 32 and the second strain relief members 34 are positioned at the apices of and connect adjacent fourth strut members about the undulating ring structure of the distal flange section 14.

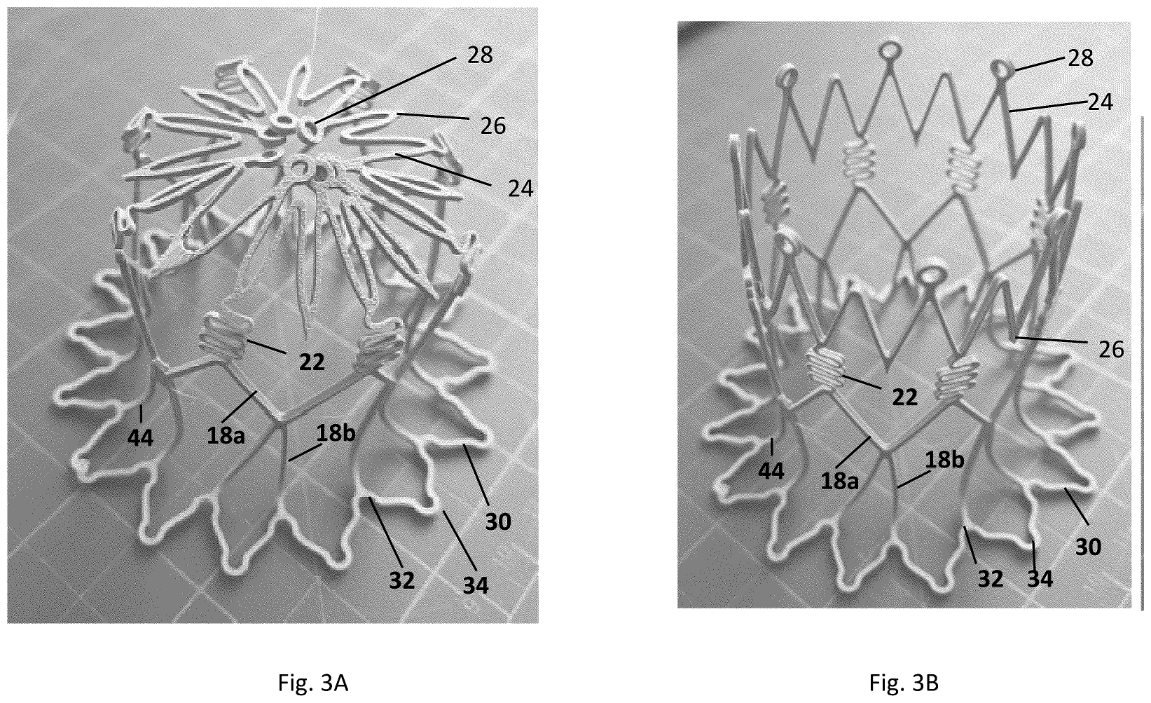

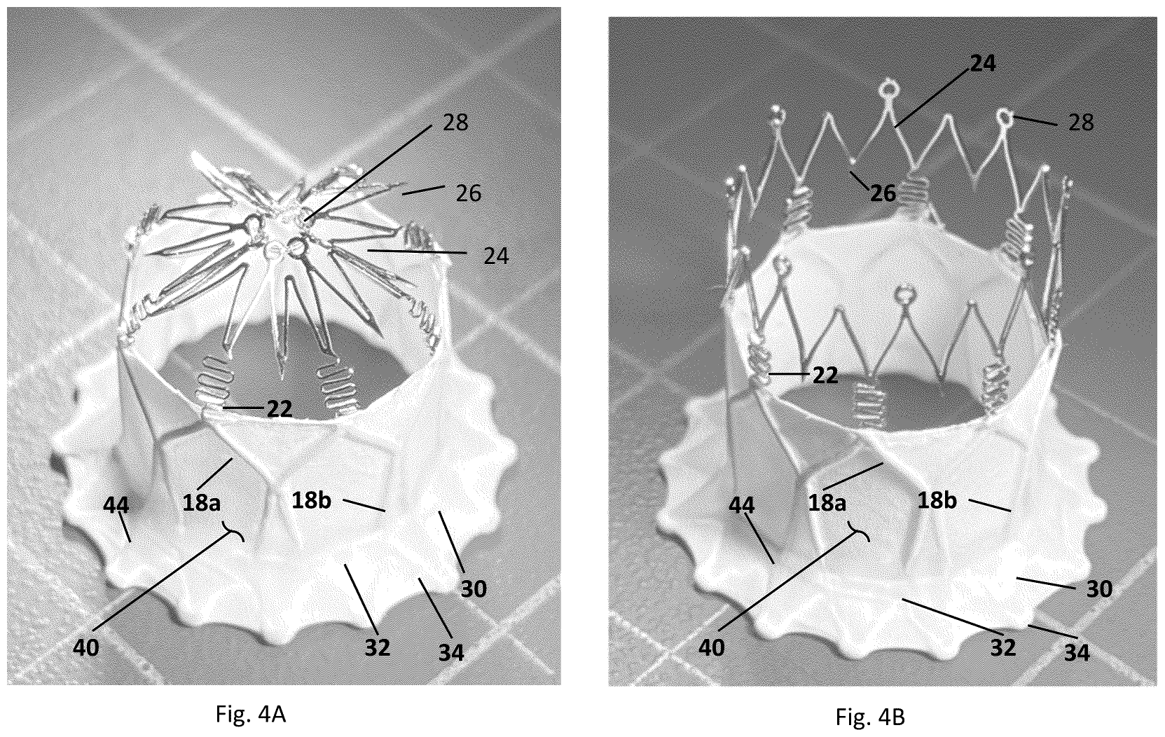

The distal flange section 14, the plurality of first strut members 18a. and the plurality of second strut members 18b are each configured to radially expand substantially independently of the proximal end section 16. In this manner, the proximal end section 16 may be independently constrained relative to the distal flange section 14 and the distal end section 17, in such a manner as to at least substantially occlude the central lumen of the tissue connection stent 10 as illustrated in FIGS. 3A, 4A, and 5A.

The proximal end section 16 may be constrained in a radially inward orientation to at least substantially occlude the central lumen of the tissue connection stent 10 in a number of manners. One particular way of constraining the proximal end section 16 is to have a purse string tie 45, such as a purse string suture, passing through the plurality of eyelets 28 and cinching the proximal end section 16 to occlude the central lumen of the tissue connection stent at least substantially.

A plurality of connection members 22 are provided that join the first plurality of strut members 18a to at least some of the plurality of third strut members 24 along a longitudinal axis of the tissue connection stent 10. The plurality of connection members 22 are longitudinally compliant and capable of flexing both longitudinally and circumferentially relative to the tissue connection stent. This compliance of the plurality of connection members 22 facilitates the radially inward deformation of the plurality of third strut members 24 to occlude the central lumen of the tissue connection stent, at least substantially, to reduce or eliminate fluid flow through the central lumen of the tissue connection stent. The connection members may be a wide variety of configurations, however, as illustrated in the accompanying Figures, one example of such connection member 22 configuration is a serpentine structure in which a proximal end of the connection member 22 is joined to the distal apex 25 of some of the plurality of third strut members 24 and a distal end of the connection member 22 is joined to an apex 19 at the proximal end of the plurality of first strut members.

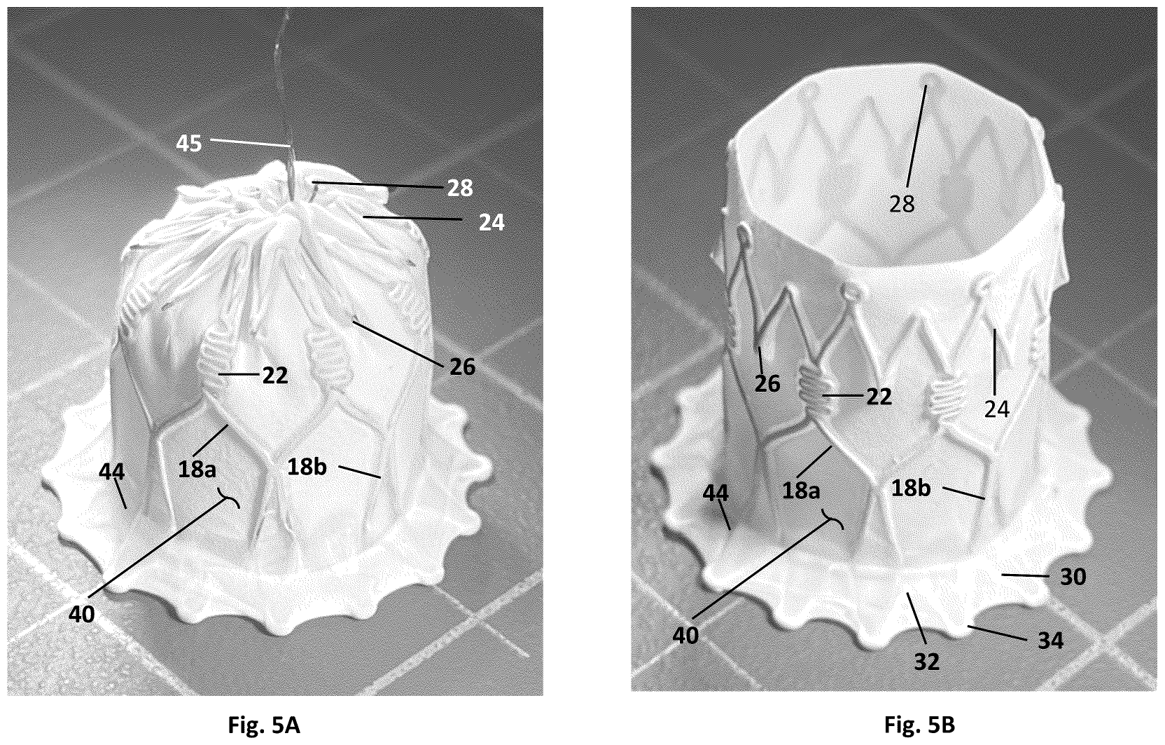

Optionally, a plurality of barbs 26 are provided at distal apices 23 of at some of the plurality of third strut members 24 that are not joined to the connection members 22. When deployed within a tubular graft 100, plurality of barbs 26 are configured to embed into the luminal wall of the tubular graft to assist in positionally securing the tissue connection stent 10 with the tubular graft. Where, as illustrated in FIGS. 5A and 5B, the proximal end section 16 of the tissue connection stent 10 is covered, the plurality of barbs 26 will cause the covering 40 to project radially outward and will also either embed into or exert focal pressure against the luminal wall of the tubular graft (now shown) to positionally secure the tissue connection stent 10 within the tubular graft.

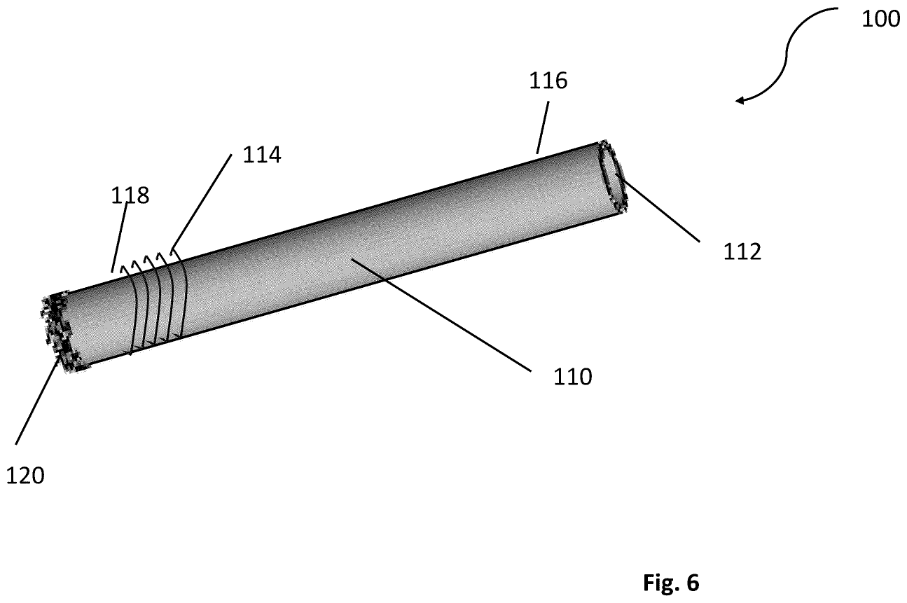

An exemplary tubular graft 100 is illustrated in FIG. 6. As described in the '495 Publication, a tubular graft assembly 100 has a plurality of tines 120 that project distally from a distal end of the tubular graft 110 as further described in the '495 Patent Publication, incorporated by reference. A central lumen 112 passes the entire length of the tubular graft 110 and is open at both proximal and distal ends of the tubular graft 110. Unlike the tubular graft described in the '495 Publication, however, tubular graft assembly 100 also includes a reinforced distal section 114 of the tubular graft 110. The reinforced distal section 114 may consist of a variety of different reinforcement approaches, including, without limitation, circumferential or helical reinforcing members that are either coupled to an abluminal wall surface of or integral with the wall of the tubular graft 110, circumferential or helical corrugations or pleats in the wall of the tubular graft 110, longitudinally, helically, or circumferentially oriented regions of the wall of the tubular graft 110 that have a higher density than remaining regions of the wall of the tubular graft 110.

The reinforced distal section 114 of the tubular graft 110 is configured to allow the distal portion of the tubular graft 110 to flex in any direction about its circumference without kinking and maintain patency of the central lumen 112. Thus, during an implant procedure, the distal end of the tubular graft 110 will be coupled to an abluminal wall of an anatomic passageway, such as by the plurality of tines 120, and will, therefore, be abutting the abluminal wall of the anatomic passageway. The tubular graft 110 will initially be oriented substantially perpendicular to the anatomic passageway. This orientation is, however, sub-optimal for coupling the proximal end of the tubular graft 110 to another conduit, such as, for example, a conduit from or to a ventricular assist device, as the proximal end of the tubular graft 110 will typically be tunneled within the body and must, therefore, lie within the sagittal or coronal planes of the body. Accordingly, the tubular graft 110 must traverse a bend between the distal end and the proximal end without kinking that partially or fully occludes the central lumen 114 of the tubular graft 110. By providing the reinforced distal section 114, the tubular graft 110 is able to be positioned with a bend and have the central lumen 114 traverse from a perpendicular orientation at the anatomic passageway to up to or greater than a ninety (90) degree bend while maintaining fluid flow patency of the central lumen 114.

It will be understood that the various embodiments described above are intended to be interchangeable with one another. For example, the configuration or orientation of the tine ring, the tines projecting distally from the tine ring, the configuration or orientation of the coring knife or knives, the configuration or orientation of the stabilization tines, the configuration or orientation of the tine ring engagements, as described above with respect to the various embodiments thereof are all intended to be and considered to be interchangeable with one another. Accordingly, the embodiments are not intended to be limited to the specific embodiments depicted in the accompanying Figures but may exchange or substitute components from other embodiments of assembly 10 or its various components.

As noted above, the variants of end-to-side anastomosis assembly 10, 60, and/or 100 may also be used in a wide variety of non-vascular medical applications to create end-to-side conduits between anatomic passageways or between a tubular conduit and an anatomic passageway. Those skilled in the art will appreciate and understand that the scope of utility and the scope of the constructs of the end-to-side assemblies of the present disclosure described herein may have a large number of variations and that the scope of the invention is limited only by the claims appended hereto.

1. A tissue connection stent, comprising a lattice framework defining a proximal tubular section, a distal flange section, wherein the lattice framework has a first diametrically reduced state and a second diametrically expanded state, the distal flange section comprising a plurality of radially projecting members arrayed about substantially an entire circumferential axis of the tissue connection stent, and the proximal tubular section comprising a proximal end section configured to occlude a central lumen of the tissue connection stent when the proximal tubular section and the distal flange section are in the second diametrically expanded state.

2. The tissue connection stent of claim 1, wherein the proximal tubular section further comprises a plurality of first strut members and a plurality of second strut members, and a plurality of connection points at apices of each of the plurality of first strut members and the plurality of second strut members.

3. The tissue connection stent of claim 2, wherein the second strut members terminate at a distal end thereof in a connection to the plurality of radially projecting members of the distal flange section.

4. The tissue connection stent of claim 1, wherein the plurality of radially projecting members further form an undulating ring having strain relief sections at proximal and distal ends of the undulating ring.

5. The tissue connection stent of claim 2, further comprising a plurality of undulating connection member connecting an apex of the plurality of second strut members with an apex of the plurality of the first strut members.

6. The tissue connection stent of claim 5, wherein each of the plurality of undulating connection members are oriented along a longitudinal axis of the tissue connection stent.

7. The tissue connection stent of claim 6, wherein each of the plurality of undulating connection members are axially flexible thereby allowing the proximal end section to radially deflect between the first diametrically reduced state and the second diametrically expanded state independently of the plurality of second strut members, the plurality of first strut members, and the distal flange section.

8. The tissue connection stent of claim 7, wherein the proximal end section further comprises a plurality of interconnected third strut members, at least some of the third strut members having an apex joined to the plurality of undulating connection members and at least some of the third strut members further comprising an eyelet at a proximal end thereof.

9. The tissue connection stent of claim 8, further comprising a plurality of barbs projecting distally from at least some of the plurality of third strut members.

10. The tissue connection stent of claim 1, further comprising a polymer covering.

11. The tissue connection stent of claim 9, wherein the polymer covering is present on the proximal tubular section and the distal flange section.

12. The tissue connection stent of claim 9, wherein the polymer covering is present on the entire tissue connection stent.

13. The tissue connection stent of claim 12, further comprising a purse string passing through a plurality of eyelets at a proximal end of the proximal end section.

14. The tissue connection stent of claim 13, wherein the purse string and the plurality of eyelets are configured to radially deflect the proximal end section between the first diametrically reduced state and the second diametrically expanded state independently of the plurality of second strut members, the plurality of first strut members, and the distal flange section.

15. The tissue connection stent of claim 14, wherein the plurality of barbs project radially outward when the proximal end section is in the first diametrically reduced state.

16. An end-to-side anastomosis system, comprising:
a. a graft having a central lumen through which the tissue connection stent is passed, the graft further comprising a flexible circumferentially and/or longitudinally reinforced distal section configured to allow the distal section of the graft to flex without kinking;
b. a plurality of securement barbs coupled to the distal end of the graft and configured to penetrate into an abluminal surface of an anatomic passageway and secure the graft to the anatomic passageway; and
c. a tissue connection stent configured to pass into the central lumen of the graft and diametrically expand and seat within the distal end of the graft, the tissue connection stent comprising a lattice framework defining a proximal tubular section, a distal flange section, wherein the lattice framework has a first diametrically reduced state and a second diametrically expanded state, the distal flange section comprising a plurality of radially projecting members arrayed about substantially an entire circumferential axis of the tissue connection stent, and the proximal tubular section comprising a proximal end section configured to occlude a central lumen of the tissue connection stent when the proximal tubular section and the distal flange section are in the second diametrically expanded state.

17. The end-to-side anastomosis system of claim 16, wherein the flexible circumferentially and/or longitudinally reinforced distal section further comprises a plurality of circumferential rib.

18. The end-to-side anastomosis system of claim 16, wherein the flexible circumferentially and/or longitudinally reinforced distal section further comprises a plurality of pleats.

19. The end-to-side anastomosis system of claim 16, wherein the graft is polymeric and the flexible circumferentially and/or longitudinally reinforced distal section further comprises regions of higher density and regions of lower density in the graft polymer.

20. The end-to-side anastomosis system of claim 16, wherein the graft polymer is selected from the group of polytetrafluoroethylene and thermoplastic polyurethane.
